(19) United States
(12) Patent Application Publication
(10) Pub. No.: US 2012/0070799 A1
(43) Pub. Date: Mar. 22, 2012

(54) Method and apparatus for reducing pain and patient discomfort at an injection site

(76) Inventor: Mark Metcalf, Newport Beach, CA (US)

(21) Appl. No.: 12/924,089

(22) Filed: Sep. 21, 2010

(51) Int. Cl. A61C 3/00; A61M 5/42

(52) U.S. Cl. 433/118; 604/112; 604/506

(57) ABSTRACT

An apparatus and a method for reducing pain and the discomfort experienced by a patient as a consequence of a needle stick during the administration of an injection. The pain-reducing apparatus includes a handle and a disposable fork detachably connected thereto. The fork has a pair of outstretched fingers that are separated from one another by a gap at which the injection site is located. Vibrations applied to the fork from the handle are transmitted from the fingers of the fork to the patient's tissue or skin surrounding the injection site. The vibrations desensitize the patient's tissue/skin during the administration of the injection.

### BACKGROUND OF THE INVENTION

1. Field of the Invention

An apparatus and a method are disclosed by which a vibrating fork applies vibrations to a tissue area surrounding the site at which an injection is to be administered to a patient. The vibrations reduce pain and minimize the discomfort suffered by the patient as a consequence of a needle stick which the injection is being administered.

2. Background Art

A syringe is used to administer an injection to a patient for a variety of reasons. For example, a medication or an anesthetic is delivered to a patient via a needle which penetrates the patient's tissue/skin at the injection site. Even the slightest pain associated with a needle stick during an injection can distress the patient. In this case, the patient may elect to forego the injection altogether, whereby an important treatment could be missed.

Accordingly, it would be desirable to be able to desensitize the injection site in order to reduce pain and minimize the discomfort that will be suffered by a patient as a consequence of an injection and the needle stick associated therewith.

### SUMMARY OF THE INVENTION

In general terms, an apparatus and a method are disclosed for reducing pain and the discomfort experienced by a patient as a consequence of a needle stick during the administration of an injection. In particular, vibrations are applied around the injection site to desensitize the patient's skin/tissue while the injection is being administered.

The pain-reducing apparatus includes a handle to be grasped in one hand of a medical professional. A shaft projects outwardly from the handle. A disposable fork is detachably connected to the shaft so as to be carried by the handle. The fork has a pair of outstretched prongs or fingers that are separated from one another by a gap. Vibrations generated by the handle are transmitted to the fork via the shaft. A series of lights (e.g., LEDs) are located around the handle to illuminate the injection site. One of the pair of fingers of the fork may be longer than the other to reach soft tissue areas when the injection site is located inside the mouth of the dental patient. The fork can have a variety of sizes and shapes depending upon the location of the injection site.

In operation, the handle is positioned by the medical professional so that the injection site is located in the gap between the fingers of the fork. As the fork is vibrated, the fingers are pressed against the patient's skin/tissue so that the vibrations are transferred to the injection site. The medical professional grasps a syringe in the other hand and moves the needle cannula thereof towards the injection site located in the gap between the fingers of the vibrating fork. The injection is then administered at the same time that the patient's skin/tissue is being vibrated by the vibrating fork.

In one embodiment of the invention, an opaque screen (e.g., a card) is attached to the fork so as to block the patient's line of sight to the injection site. In another embodiment, the pair of fingers of the fork are pivotally coupled together and rotatable relative to one another so as to be moved apart, whereby to stretch and tighten the skin at the injection site and thereby facilitate the administration of the injection.

### BRIEF DESCRIPTION OF THE DRAWINGS

FIG. 1 illustrates an apparatus and corresponding method according to a preferred embodiment of this invention for reducing pain and patient discomfort during the administration of an injection when the injection site is located within the patient's mouth;

FIG. 2 is an enlarged view of a vibrating disposable fork from the pain-reducing apparatus of FIG. 1;

FIG. 2A is a front view of a handle of the pain-reducing apparatus in the direction of lines 2A-2A of FIG. 2;

FIGS. 3-5 show a first alternate embodiment for the vibrating fork of FIG. 2 to be detachably connected to the handle of the pain-reducing apparatus;

FIGS. 6 and 7 show another alternate embodiment for the vibrating fork of FIG. 2;

FIGS. 8 and 9 show yet another alternate embodiment for the vibrating fork of FIG. 2;

FIG. 10 shows a card bearing decorative graphics attached to the fork of the pain-relieving apparatus in order to lie in and block the line of sight from the patient to the injection site;

FIG. 11 shows an enlarged detail of the card of FIG. 10 attached to the fork of the pain-relieving apparatus;

FIG. 12 shows a vibrating fork of the pain-relieving apparatus having a pair of rotatable fingers that are rotated towards one another;

FIG. 13 shows the pair of fingers of the vibrating fork of FIG. 12 rotated away from one another to stretch a patient's skin at an injection site; and

FIG. 14 is an exploded view of the vibrating fork.

### DESCRIPTION OF THE PREFERRED EMBODIMENT

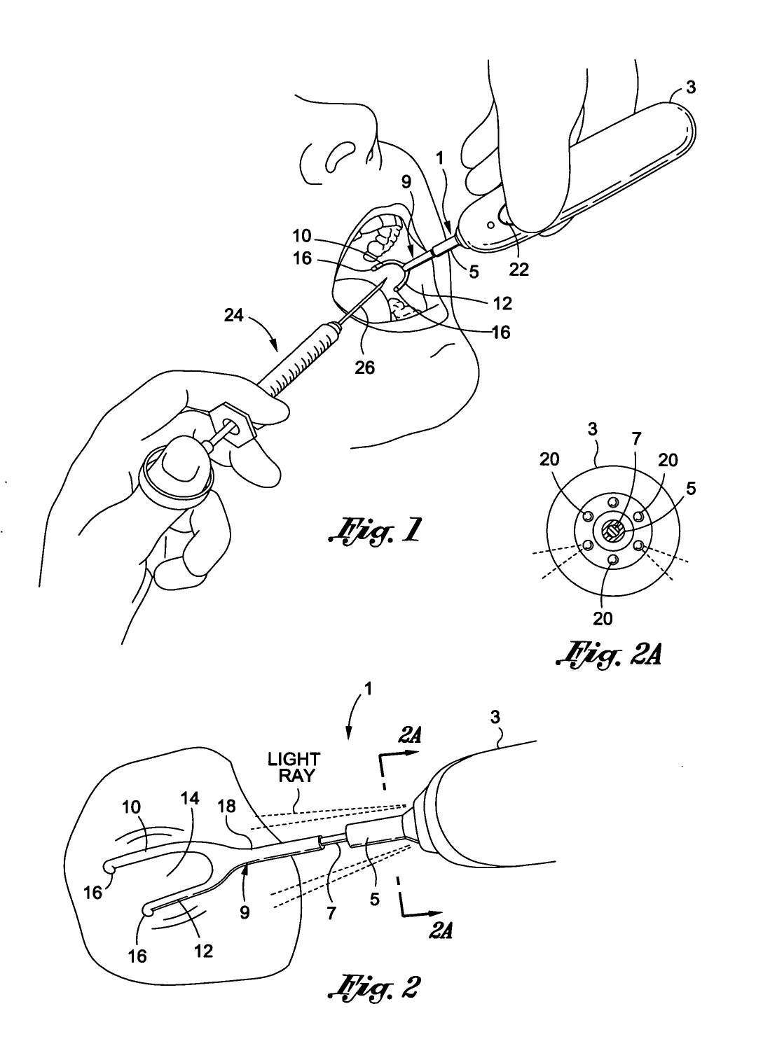

Referring initially to FIGS. 1-2A of the drawings, an apparatus and a method are described for reducing the pain of a needle stick and the discomfort associated therewith when an injection is administered to a patient. While the apparatus and method herein disclosed have particular application to the field of dentistry during the delivery of an anesthetic to the tissue inside the mouth of a patient undergoing a dental procedure, it is to be understood that this invention is advantageous to other medical fields and different injection sites regardless of the medication to be delivered.

According to a preferred embodiment, the pain-reducing apparatus 1 includes a handle 3 to be held in the hand of a medical professional (e.g., a dentist). The handle 3 shown in FIGS. 1 and 2 has a generally cylindrical configuration. However, the precise shape of the handle 3 is not to be considered a limitation of this invention. Thus, the handle 3 may have different shapes to enhance the grip and maneuverability of the vibrating pain-reducing apparatus 1 in relatively small operating areas.

A sleeve 5 projects outwardly from one end of the handle 3 of the apparatus 1. One end of a narrow shaft 7 is connected to the sleeve 5. The opposite end of the shaft 7 extends from the sleeve 5 so that a generally U-shaped fork 9 can be coupled to and carried by the handle 3 of the pain-reducing apparatus 1 by way of the shaft 7 and the sleeve 5.

The handle 3 of apparatus 1 includes a battery compartment (not shown) within which one or more batteries are stored. The batteries generate a DC voltage for causing the shaft 7 to vibrate. The vibration of the shaft 7 causes a corresponding vibration of the fork 9. As will soon be explained, the vibration of the fork 9 is transmitted to the patient's tissue at an (e.g., oral) injection site to substantially desensitize the tissue during a needle stick as a medication is administered.

The fork 9 of the pain-reducing apparatus 1 preferably includes a pair of outstretched prongs or fingers 10 and 12 that are separated from one another by a gap 14. As will soon be described, the vibrating fork 9 is positioned so that the injection site is located within the gap 14. A first end of each of the fingers 10 and 12 terminates at an outwardly protruding tip 16 (best shown in FIG. 2). Prior to the injection, the tips 16 at the ends of the fingers 10 and 12 can be moved into engagement with the patient's mouth to help hold the mouth open. When the vibrating fork 9 is pressed against the patient's tissue around the injection site, the vibrations are transferred from the fingers 10 and 12 of the fork to the tissue.

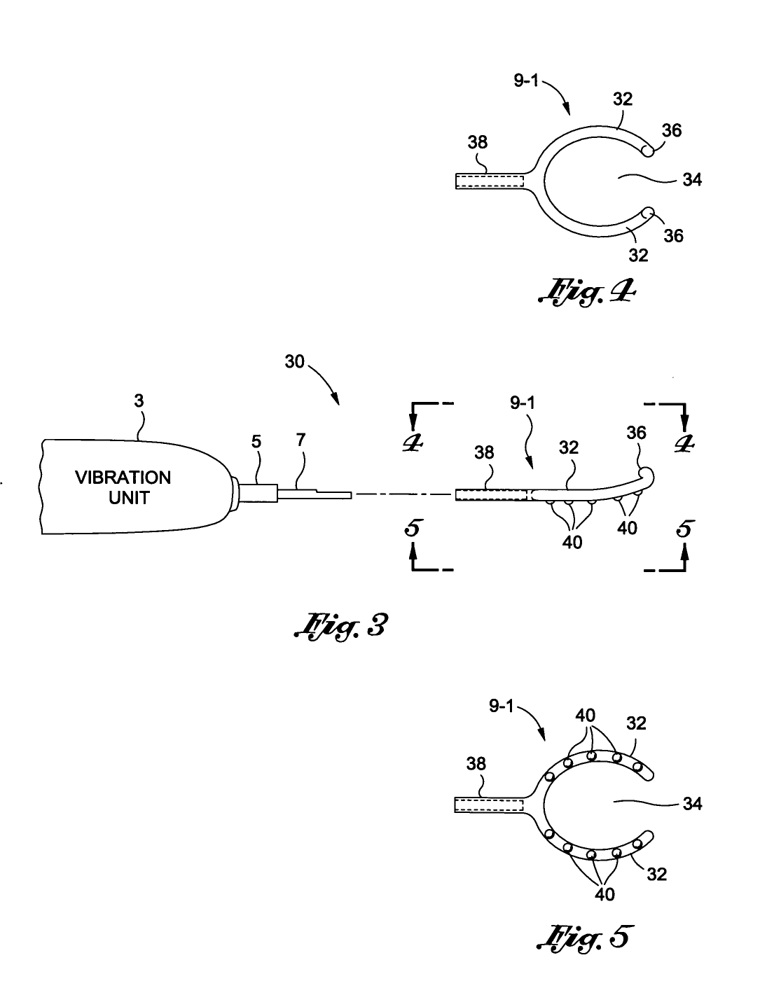

The opposite ends of the fingers 10 and 12 of fork 9 are joined to one another at a hollow stem 18. The hollow stem 18 is sized for the removable receipt therewithin of the vibrating shaft 7 that is attached to the handle 3 of pain-reducing apparatus 1. By virtue of the foregoing, the fork 9 is detachably connected to the shaft 7 so as to be disposable following a single use. In this same regard, different forks (e.g., such as those shown in FIGS. 3-9) having a variety of sizes and shapes can be detachably connected to the shaft 7 depending upon the location of the injection site.

As is best shown in FIG. 2, one of the outstretched fingers 10 of the vibrating fork 9 is longer than the other finger 12. In the case where the injection site is located inside the mouth of a dental patient, the longer finger 10 is adapted to lay against and apply vibrations to the softer tissue found at the back of the patient's mouth.

As is best shown in FIG. 2A, a series of light sources 20 (e.g., LEDs) are mounted at the front of the handle 3 of the pain-reducing apparatus 1. The light sources 20 are powered by the batteries stored within the handle 3. The light sources 20 surround the sleeve 5 of the handle 3 so that rays of light are emitted to illuminate the targeted injection site. Such illumination is particularly useful in cases (such as that shown in FIG. 1) where the injection is to be administered inside the mouth of a dental patient or at any other location where the available ambient light is limited.

In operation, and referring specifically to FIG. 1, the health care professional (i.e., dentist) grasps the handle 3 of the pain-reducing apparatus 1 in one hand so that the fork 9 is moved inside the patient's mouth. The apparatus 1 is energized (by moving an on-off power switch 22 on the handle 3 to the on position), whereby to cause the fork 9 carried by the handle 3 to vibrate. The vibrating fork 9 is pressed against the patient's tissue so that the elongated fingers 10 and 12 thereof surround the injection site within the gap 14. As previously described, the vibrations are applied to the patient's tissue by the vibrating fingers 10 and 12.

While the fork 9 is vibrating against the patient's tissue, the dentist manipulates a conventional syringe 24 with the other hand so that a needle cannula 26 is moved through the tissue at the injection site between the elongated fingers 10 and 12. It has been found that stimulating (i.e., vibrating) the tissue around the injection site during the administration of the injection minimizes pain and reduces the discomfort of the patient as a consequence of the needle stick. Shortly after the injection has been completed, the vibrating fork 9 is removed from the patient's mouth. The on-off switch 22 at the handle 3 of pain-reducing apparatus 1 is moved to the off position to terminate the vibrations and extinguish the light rays. Once the patient's mouth has been anesthetized, the dentist can perform the intended dental procedure.

As was previously disclosed, the vibrating fork can have a variety of sizes and shapes depending upon the location of the injection site. Turning in this regard to FIGS. 3-5 of the drawings, there is shown an alternate embodiment for a disposable vibrating fork 9-1 to be carried by the handle 3 of a pain-reducing apparatus 30. In this case, each of the prongs or fingers 32 of fork 9-1 has an arcuate shape. The fingers 32 are spaced from one another so as to have a “C” or horseshoe shape. A gap 34 between the fingers is sized so as to be located over the injection site. An outwardly protruding tip 36 is located at a first end of each finger 32 of fork 9-1. The opposite ends of the fingers 32 are joined together at a hollow stem 38 in which the shaft 7 of the handle 3 is removably received so that the disposable fork 9-1 is detachably connected to the shaft 7 to be carried by the handle 3. A series of massage bumps or protrusions 40 are formed on one side of the fork 9-1 opposite the side from which the tips 36 protrude. The patient's tissue is stimulated (i.e., vibrated) by the massage bumps 40 as the fork 9-1 is vibrating during the administration of an injection.

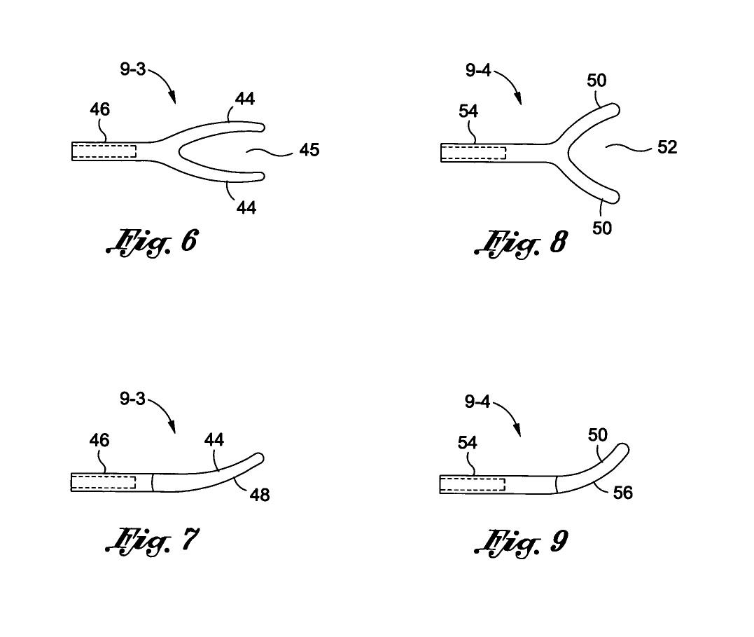

FIGS. 6 and 7 of the drawings show an additional alternate embodiment for a disposable vibrating fork 9-3 to be carried by the handle of the pain-reducing apparatus like that shown in FIG. 1. In this case, the fork 9-3 has a pair of outstretched prongs or fingers 44 that are spaced from one another in a generally V-shaped configuration. A gap 45 is located between the fingers 45 to lie over the injection site. The fingers 44 are joined together at a hollow stem 46 in which to removably receive a shaft (designated 7 in FIG. 1) so that the fork 9-3 can be coupled to and vibrated by the handle of the pain-reducing apparatus (designated 1 in FIG. 1).

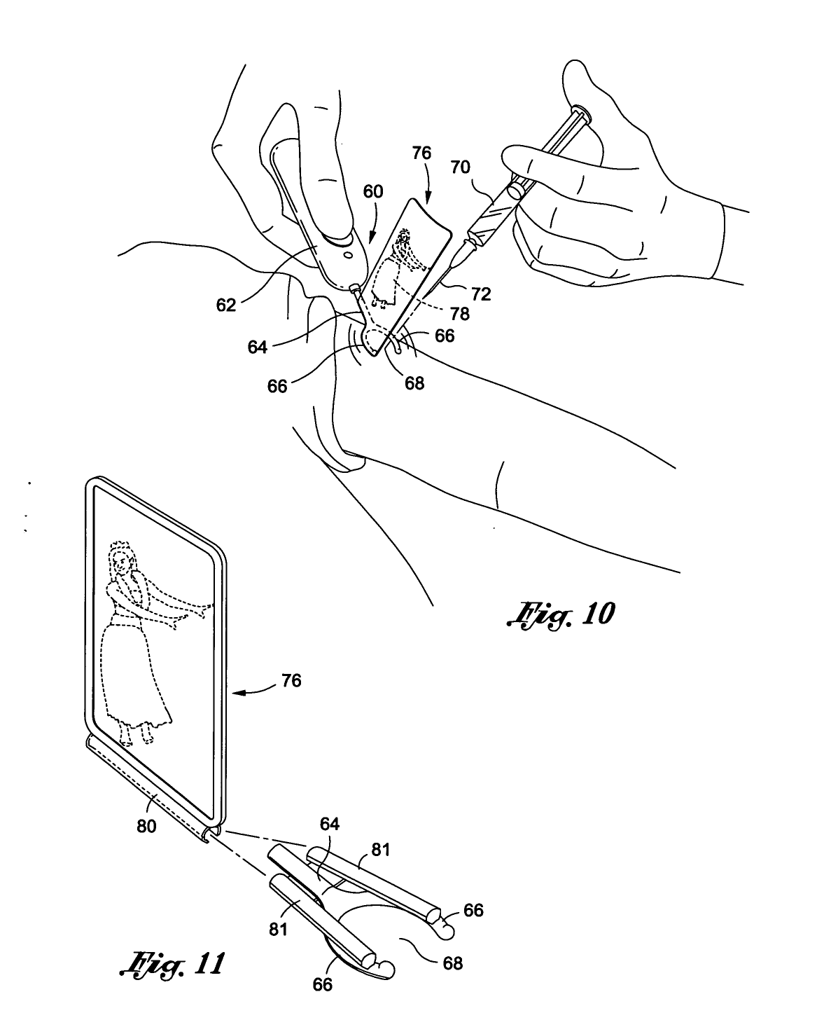

FIGS. 8 and 9 of the drawings show another alternate embodiment for a disposable vibrating fork 9-4 to be carried by the handle of the pain-reducing apparatus like that shown in FIG. 1. In this case, the fork 9-4 has a pair of outstretched prongs or fingers 50 that are spaced from one another in a generally Y-shaped configuration. A gap 52 is located between the fingers 50 to lie over the injection site. The fingers 50 are joined together at a hollow stem 54 in which to removably receive a shaft (designated 7 in FIG. 1) so that the fork 9-4 can be coupled to and vibrated by the handle of the pain-reducing apparatus (designated 1 in FIG. 1). A wide gap 52 between the fingers 50 of the Y-shaped fork 9-4 is especially useful in cases where the syringe is thrown by the healthcare professional towards the targeted injection site like a dart.

The vibrating forks 9-3 and 9-4 shown in FIGS. 6-9 have pairs of outstretched fingers 44 and 50 that bend or turn upwardly from generally-flat stems 46 and 54 at which the fingers are joined to one another. In these cases, the bottom sides 48 and 56 of the respective upturned fingers 44 and 50 of FIGS. 7 and 9 can be pressed directly against the patient's tissue to stimulate and vibrate the tissue around the injection site.

FIG. 10 of the drawings shows the vibrating pain-relieving apparatus 60 of this invention being used to stimulate a patient's tissue where the injection site is through the skin outside the mouth (e.g., at the patient's arm). The apparatus 60 includes a handle 62 and a detachable fork 64 carried by the handle 62. The fork 64 includes a pair of outstretched prongs or fingers 66 that are spaced from one another so as to surround the injection site. The healthcare professional manipulates a syringe 70 so that the needle cannula 72 thereof penetrates the patient's skin within a gap 68 between the fingers 66. As previously explained (while referring to FIGS. 1-2A), the fork 64 is vibrated, and the vibrations are transferred to the patient's skin around the injection site to reduce pain and minimize discomfort as a consequence of the needle stick.

In some situations, it would be desirable to block the injection site from the eyes of the patient, particularly in the case of a young child. To accomplish the foregoing, an opaque screen (e.g., a card 76) is mounted on the fork 64 of the pain-relieving apparatus 60. Visually pleasing graphics 78 may be printed on the card 76 in order to lie in the line of sight from the eyes of the patient to the injection site. As the fork 64 is vibrated, the card 76 is likewise vibrated to draw the attention of the patient away from the injection.

As is best shown in FIG. 11, the card 76 is removably attached to the fork 64. In this case, a channel 80 attached to the bottom of the card 76 is mated to one of a pair of supports 81 that are attached (e.g., adhesively bonded) to the fingers 66 of the fork 64. Thus, the card 76 stands upwardly from the fork 64 to hide the injection from the patient.

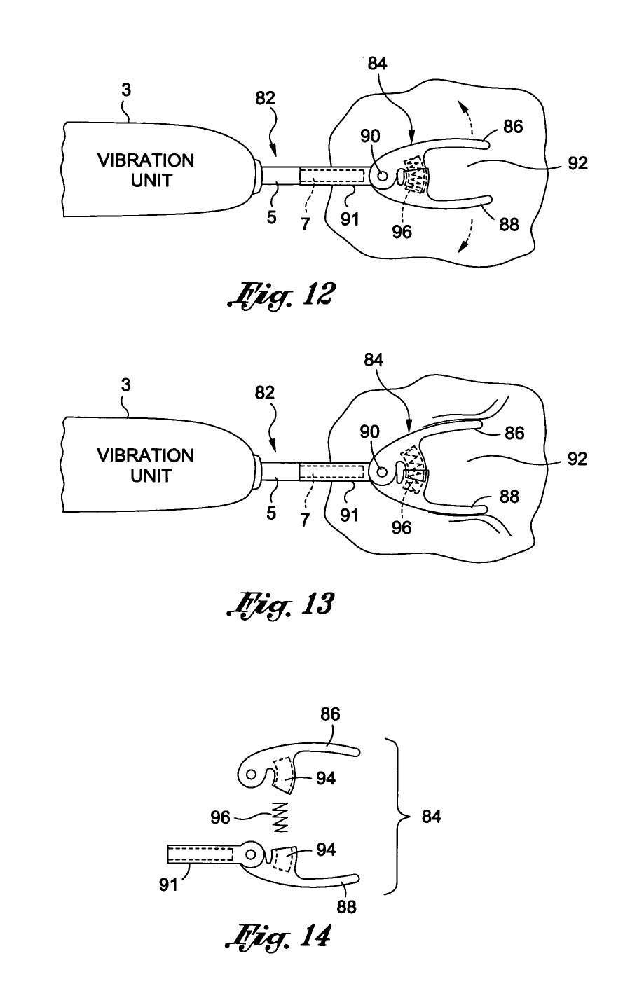

FIGS. 12-14 of the drawings show a pain-reducing apparatus 82 including a skin or tissue-stimulating disposable fork 84 having a pair of outstretched prongs or fingers 86 and 88 that are pivotally connected together and adapted to rotate relative to one another. The fork 84 is carried by a handle 3 like that shown in FIG. 1. First ends of the fingers 86 and 88 of fork 84 are coupled to each other by a pivot pin 90. A hollow stem 91 (best shown in FIG. 14) extends from the first end of one of the fingers (e.g., 88) to removably receive therewithin the shaft 7 of the handle 3, whereby the fork 84 is detachably connected to the shaft 7. The opposite ends of the fingers 86 and 88 of the fork 84 are spaced from each other so that a gap 92 is established therebetween. The size of the gap 92 between the fingers 86 and 88 is expandable in a manner that will now be disclosed.

Each of the pair of fingers 86 and 88 of the fork 84 has a spring pocket 94 (also best shown in FIG. 14) for receipt of a spring 96. The opposite ends of the spring 96 are fixedly retained within respective ones of the spring pockets 94. The spring pockets 94 are preferably sized so that one spring pocket is slidable within the other. FIG. 12 shows the pain-reducing apparatus 82 when the fork 84 is first laid against the patient's skin or tissue whether outside or inside the mouth so that the injection site is located in the gap 92 between fingers 86 and 88. In this case, the spring 96 is relaxed within the spring compartments 94 and the fingers 86 and 88 are rotated together.

When the fork 84 is pressed down against the patient's skin or tissue as shown in FIG. 13, the fingers 86 and 88 are caused by the pressure of the skin or tissue to rotate at pivot pin 90 so as to be pushed apart from one another. The spring 96 is now stretched so as to store energy. At the same time, the patient's skin or tissue will be stretched and tightened at the injection site within the gap 92 between the fingers 86 and 88. Tightening the patient's skin or tissue in this manner makes the administration of the injection easier for the healthcare professional. In this same regard, the vibrations transmitted to the skin or tissue as the fork 84 is vibrated reduces pain and minimizes patient discomfort during the injection. At the conclusion of the injection, the fork 84 is lifted off the patient's skin or tissue, whereby the spring 96 contracts and releases its stored energy to automatically pull the fingers 86 and 88 towards one another.

Although this invention has been described as reducing pain at an injection site, the teaching of this invention are also applicable to treatment areas of the patient's skin or tissue that are not subjected to a needle stick. For example, the pain-reducing apparatus herein disclosed can also be used to desensitize the skin or tissue from a stinging or burning sensation that occurs when certain medications are topically applied without a syringe.

1. A method for reducing pain experienced by a patient during the administration of an injection, said method comprising the step of massaging the skin of the patient adjacent the injection site for desensitizing the skin to a needle stick.

2. The method recited in claim 1, comprising the additional step of massaging the patient's skin around the injection site.

3. The method recited in claim 1, comprising the additional step of massaging the patient's skin by applying vibrations to the skin adjacent the injection site.

4. The method recited in claim 3, comprising the additional steps of applying the vibrations to the patient's skin by means of pain-reducing apparatus including a handle to be grasped in the hand of a healthcare professional and at least one finger extending from said handle; moving the finger against the patient's skin; and causing the finger to vibrate so that the vibrations are transmitted from the finger to the skin adjacent the injection site.

5. The method recited in claim 4, wherein said pain-reducing apparatus includes a pair of fingers extending from said handle where said fingers are separated from one another by a gap, said method comprising the additional steps of moving said pair of fingers against the patient's skin such that the injection site is located in the gap between the fingers; and causing the pair of fingers to vibrate so that the vibrations are transmitted from the fingers to the skin adjacent the injection site.

6. The method recited in claim 5, comprising the additional steps of forming at least one massage bump protruding outwardly from each of said pair of fingers; and pressing said pair of fingers against the patient's skin such that the vibrations are transmitted from the fingers to the skin by the massage bump of each finger.

7. The method recited in claim 5, comprising the additional steps of forming a series of massage bumps projecting outwardly from each of said pair of fingers; and pressing said pair of fingers against the patient's skin such that the vibrations are transmitted from the fingers to the skin by the series of massage bumps of each finger.

8. The method recited in claim 5, comprising the additional step of making one of said pair of fingers longer than the other.

9. The method recited in claim 5, comprising the additional step of mounting a screen on said pain-reducing apparatus to block the line of sight from the patient to the injection site.

10. The method recited in claim 5, comprising the additional step of coupling said pair of fingers together such that said fingers are rotatable away from one another, whereby to increase the size of said gap therebetween.

11. The method recited in claim 10, comprising the additional step of connecting a spring between said pair of fingers, said spring being stretched and storing energy when said fingers are rotated away from one another.

12. The method recited in claim 5, wherein said pair of fingers are detachably connected to said handle, said method comprising the additional steps of detaching said fingers from said handle following the administration of the injection; and discarding said fingers.

13. Apparatus for reducing pain experienced by a patient during the administration of an injection, said apparatus comprising:
a handle to be grasped by a healthcare professional; and
at least one finger carried by said handle,
said handle adapted to cause said one finger to vibrate and to enable said one finger to be positioned against the patient's skin, whereby the vibrations of said finger are transmitted to the patient's skin adjacent the injection site to desensitize the skin to a needle stick.

14. The apparatus recited in claim 13, wherein said one finger is detachably connected to said handle so as to be detachable from said handle for disposal following the administration of the injection.

15. The apparatus recited in claim 13, wherein said apparatus comprises a pair of fingers carried by and caused to vibrate by said handle, said fingers being spaced from each other by a gap between said fingers, said gap being sized so that the injection site is located in said gap.

16. The apparatus recited in claim 15, wherein one of said pair of fingers is longer than the other.

17. The apparatus recited in claim 15, wherein each of said pair of fingers has at least one massage bump protruding therefrom so that the vibrations of said fingers are transmitted to the patient's skin adjacent the injection site at the massage bump of each finger.

18. The apparatus recited in claim 15, further comprising a screen attached to at least one of said pair of fingers in order to block the line of sight from the patient to the injection site during the administration of the injection.

19. The apparatus recited in claim 15, wherein said pair of fingers are pivotally coupled together so as to be rotatable away from one another whereby to increase the size of said gap between said fingers.

20. The apparatus recited in claim 20, further comprising a spring connected between said pair of fingers, said spring being stretched to store energy when said fingers are rotated away from one another.
